(19) United States
(12) Patent Application Publication
(10) Pub. No.: US 2008/0241943 A1
(43) Pub. Date: Oct. 2, 2008

(54) Subnanomolar Precipitator of Thiophilic Metals

(75) Inventors: Kim D. Janda, La Jolla, CA (US); Tobin J. Dickerson, San Diego, CA (US); James J. La Clair, San Diego, CA (US)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(73) Assignee: The Scripps Research Institute, La Jolla, CA (US)

(21) Appl. No.: 11/791,128

(22) PCT Filed: Nov. 18, 2005
(86) PCT No.: PCT/US05/42081

§ 371 Date: Apr. 29, 2008

Related U.S. Application Data
(60) Provisional application No. 60/629,687, filed on Nov. 19, 2004.

(51) Int. Cl. G01N 33/20; C07D 333/72; C07D 333/50; C07D 409/02

(52) U.S. Cl. 436/73; 549/52; 549/41; 549/3

(57) ABSTRACT

A fluorescent dye-doped crystalline assay is employed for selection and detection of thiophilic heavy metal ions. While comparable in analytical performance to known solution based methodologies, the formation of crystalline analytes provides for signal amplification, and consequently, a powerful platform whose analysis is directly amenable to high-throughput video capture systems. In a microcapillary format, this assay is capable of screening hundreds of samples per day for the presence of subnanomolar concentrations of Hg²⁺ using a conventional fluorescence microscope.

### FIELD

The invention relates to the selection, precipitation, detection, and isolation of heavy metals and heavy metal ions. More particularly, the invention relates to dithiophthalide ligands and to their use for selecting, precipitating, detecting and isolating thiophilic-metals.

### BACKGROUND

The detection of toxic metals, including mercury and lead, has become a vital analytical tool for environmental remediation and regulation of food stocks. The discovery that aquatic organisms convert elemental mercury to methylmercury, which subsequently concentrates through the food chain in the tissues of fish and marine mammals has added an urgency to this need (Nendza, M.; et al. Chemosphere 1997, 35, 1875-1885). A prevalent obstacle with the current assessment of metal ion contamination originates from the lack of adequate assay throughput. In this context, a critical concern with current analyses stems from the fact that the majority of these assays are solution-based and thus the response is highly dependent upon assay environment.

A plethora of indicators are known to detect metal ions through the intramolecular modulation of a pendant chromophore (Choi, M. J.; et al. Chem. Commun. 2001, 1664-1665; Brümmer, O.; et al. Org. Lett. 1999, 1, 415-418; Sancenón, F.; et al. Chem. Commun., 2001, 2262-2263; F. Sancenón, F.; et al. Tetrahedron Lett. 2001, 42, 4321-4323; Moon, S. Y.; et al. J. Org. Chem. 2004, 69, 181-183; Palomares, E.; et al. Chem. Commun. 2004, 362-363; Kuhn, M. A.; et al. Proc. SPEI, Intl. Soc. Opt. Eng. 1995, 2388, 238-242). While effective for toxic metal analysis in laboratory environments (Baeumner, A. J. Anal. Bioanal. Chem., 2003, 377, 434-445; Epstein, J. R.; Walt, D. R. Chem. Soc. Rev., 2003, 32, 203-214; Jain, K. K. Med. Device Technol., 2003, 14, 10-15; Ulber, R.; et al. Anal. Bioanal. Chem. 2003, 376, 342-348), few of these probes have proven suitable for screening within modern industrial settings (Roblin, P.; Barrow, D. A. J Environ Monit., 2000, 2, 385-392). While the design of colorimetric-ligation has been critically-evaluated by Lippard, Haugland and others (Descalzo, A. B.; et al. J. Am. Chem. Soc., 2003, 125, 3418-3419; Nolan, E. M.; et al. J. Am. Chem. Soc., 2003, 125, 14270-14271; Xu, X.; et al. Anal Chem. 2002, 74, 3611-3615; Zhang, X. B.; et al. Anal Chem. 2002, 74, 821-825), the predominant theme within metal ion sensing relies on solution based chemistry. Few efforts have correlated metal ion sensing with material synthesis (i.e., crystallization, precipitation or polymerization).

Recent advances in the study of calorimetric ligands suggest that a viable metal indicator offers: 1) the appropriate direction of metal-mediated modulation, 2) a high degree of sensitivity, and 3) a defined metal selectivity. We have demonstrated that correlation of ligation-induced calorimetric response with precipitation provides an effective vector for metal ion analysis (Brümmer, O.; et al. Org. Lett. 1999, 1, 415-418). Here amplification of a calorimetric response through precipitation provided a practical screen for mercuric ion.

Unquestionably, the development of facile and efficient methodologies for the detection of heavy metals in a wide range of settings is paramount in the effort to minimize incidences of mortality attributable to heavy metal toxicity. What is needed is a showing that the appendage of a calorimetric moiety is unnecessary for the analysis of thiophilic metals and adapt this to provide a digital screen for the analysis of Hg²⁺, Pb²⁺ and Cd²⁺.

### SUMMARY

A fluorescent dye-doped crystalline assay is disclosed that offers convincing metal selection and provides detection comparable to conventional solution-based ligands used for the spectrofluorometric analysis of thiophilic heavy metal ions. While comparable in analytical performance to known methodologies, the formation of a crystalline analytes provides for signal amplification, and consequently, a powerful platform whose analysis is directly amenable to high-throughput video capture systems. This procedure has been tested in a variety of scenarios and shows good performance using readily available equipment, including a commercially available USB CCD camera. Furthermore, when employed in a microcapillary format, this assay is capable of screening hundreds of samples per day for the presence of subnanomolar concentrations of Hg²⁺ using a conventional fluorescence microscope.

One aspect of the invention is directed to a thiophilic metal-ligand complex represented by formula I:

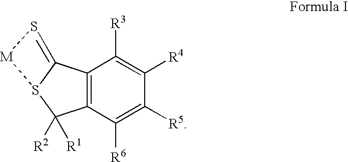

In the above Formula I, M is a multivalent heavy metal ion; R¹ and R² are each radicals independently selected from the group consisting of C1-C10 alkyl, C6-C10 aryl, C5-C10 heteroaryl, and a radical represented by the following structure:

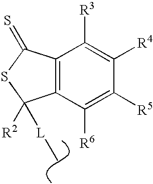

or alternatively, R¹ and R² together form a diradical represented by formula II:

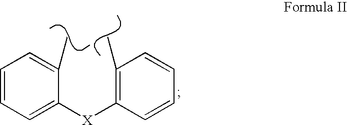

R³, R⁵, and R⁵ are each radicals independently selected from the group consisting of hydrogen, —NO₂, —NH₂, —OH, —CO₂H, —CO₂Me, —CO₂t-Bu, —CH₂OH, —CHO, —C(O)CH₃, —Cl, —Br, —I, —CF₃, —CN, —CH═CH₂, C1-C10 alkyl, C6-C10 aryl, and C5-C10 heteroaryl; and R⁴ is a radical selected from the group consisting of hydrogen, —NO₂, —NH₂, —OH, —CO₂H, —CO₂Me, —CO₂t-Bu, —CH₂OH, —CHO, —C(O)CH₃, —Cl, —Br, —I, —CF₃, —CN, —CH═CH₂, C1-C10 alkyl, C6-C10 aryl, C5-C10 heteroaryl, and either radical represented by the following structures:

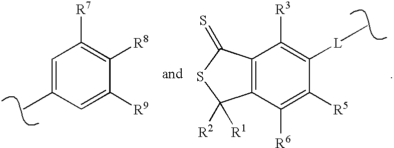

In the above structures, R⁷, R⁸, and R⁹ are each radicals independently selected from the group consisting of hydrogen, —NO₂, —NH₂, —OH, —CO₂H, —CO₂Me, —CO₂t-Bu, —CH₂OH, —CHO, —C(O)CH₃, —Cl, —Br, —I, —CF₃, —CN, —CH═CH₂, C1-C10 alkyl, C1-C10 aryl, and C5-C10 heteroaryl; X is a diradical selected from the group consisting of —O—, —NH—, —C(O)—, and —C(S)—; and L is a diradical selected from the group of diradicals consisting of C1-C10 alkyl, C6-C10 aryl, C5-C10 heteroaryl, polyethylene glycol having 1-10 subunits, peptide having 1-10 amino acid residues, and oligonucleotide having 1-10 nucleotide residues. In a preferred embodiment of this aspect of the invention, M is a multivalent metal ion selected from the group consisting of Hg⁺⁺, Pb⁺⁺, Cd⁺⁺, Au⁺⁺⁺, Cu⁺⁺, Pt⁺⁺, Pd⁺⁺, Ni⁺⁺, Co⁺⁺, and Mo⁺⁺. In another preferred embodiment of this aspect of the invention, R⁴ is a radical selected from the group consisting of —H, —Cl, —Br, and —I. In another preferred embodiment of this aspect of the invention, R⁸ is a radical selected from the group consisting of —H, —Cl, —Br, and —I. A preferred subgenus of the invention is represented by the following structure:

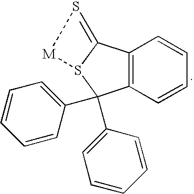

Another preferred subgenus of the invention is represented by the following structure:

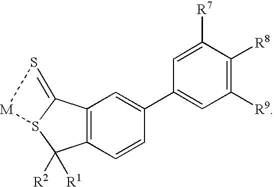

In a preferred embodiment of the above subgenus, R⁷ and R⁹ are hydrogen and R⁸ is a radical selected from the group consisting of —H, —Cl, —Br, and —I. Another preferred subgenus of the invention is represented by the following structure:

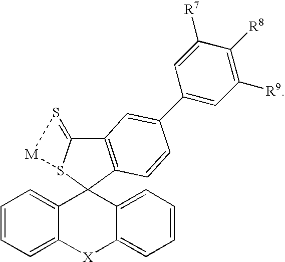

In a preferred embodiment of the above subgenus, R⁷ and R⁹ are hydrogen and R⁸ is a radical selected from the group consisting of —H, —Cl, —Br, and —I. Another preferred subgenus of the invention is represented by the following structure:

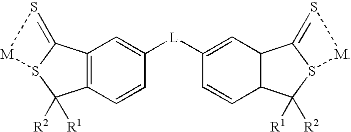

Another preferred subgenus of the invention is represented by the following structure:

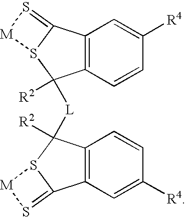

Another aspect of the invention is directed to a precipitate of any of the thiophilic metal-ligand complexes described above.

Another aspect of the invention is directed to a dithiophthalide ligand represented by formula III:

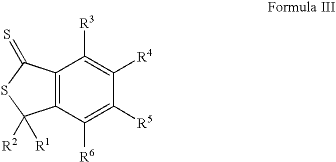

In Formula II, R¹ and R² are each radicals independently selected from the group consisting of C1-C10 alkyl, C6-C10 aryl, C5-C10 heteroaryl, and a radical represented by the following structure:

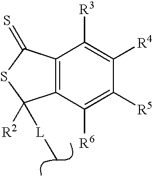

or alternatively, R¹ and R² together form a diradical represented by formula IV:

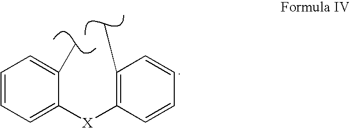

In Formula IV, R³, R⁵, and R⁶ are each radicals independently selected from the group consisting of hydrogen, —NO₂, —NH₂, —OH, —CO₂H, —CO₂Me, —CO₂t-Bu, —CH₂OH, —CHO, —C(O)CH₃, —Cl, —Br, —I, —CF₃, —CN, —CH═CH₂, C1-C10 alkyl, C6-C10 aryl, and C5-C10 heteroaryl; and R⁴ is a radical selected from the group consisting of hydrogen, —NO₂, —NH₂, —OH, —CO₂H, —CO₂Me, —CO₂t-Bu, —CH₂OH, —CHO, —C(O)CH₃, —Cl, —Br, —I, —CF₃, —CN, —CH═CH₂, C1-C10 alkyl, C6-C10 aryl, C5-C10 heteroaryl, and either radical represented by the following structures:

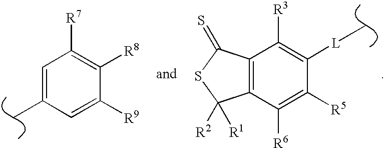

In the above structures, R⁷, R⁸, and R⁹ are each radicals independently selected from the group consisting of hydrogen, —NO₂, —NH₂, —OH, —CO₂H, —CO₂Me, —CO₂t-Bu, —CH₂OH, —CHO, —C(O)CH₃, —Cl, —Br, —I, —CF₃, —CN, —CH═CH₂, C1-C10 alkyl, C6-C10 aryl, and C5-C10 heteroaryl; X is a diradical selected from the group consisting of —O—, —NH—, —C(O)—, and —C(S)—; and L is a diradical selected from the group of diradicals consisting of C1-C10 alkyl, C6-C10 aryl, C5-C10 heteroaryl, polyethylene glycol having 1-10 subunits, peptide having 1-10 amino acid residues, and oligonucleotide having 1-10 nucleotide residues. However, the following provisos apply: R⁴ is hydrogen only if R¹ or R² is a radical represented by the following structure:

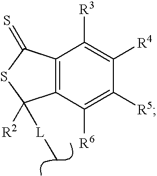

or alternatively, if R¹ and R² together for a diradical represented by formula II:

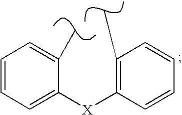

and R⁴ and R⁵ can not together form a diradical. In a preferred embodiment of this aspect of the invention, R⁴ is a radical selected from the group consisting of —H, —Cl, —Br, and —I. In another preferred embodiment of this aspect of the invention, R⁸ is a radical selected from the group consisting of —H, —Cl, —Br, and —I. In a subgenus of this aspect of the invention, the dithiophthalide ligand is represented by the following structure:

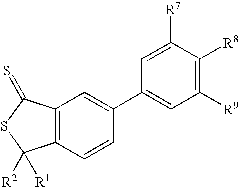

In a preferred embodiment of this subgenus, R⁷ and R⁹ are hydrogen and R⁸ is a radical selected from the group consisting of —H, —Cl, —Br, and —I. In another subgenus of this aspect of the invention, the dithiophthalide ligand is represented by the following structure:

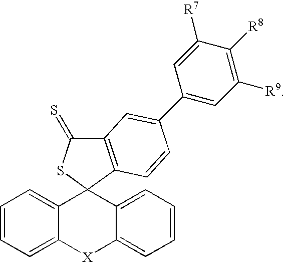

In a preferred embodiment of this subgenus, R⁷ and R⁹ are hydrogen and R⁸ is a radical selected from the group consisting of —H, —Cl, —Br, and —I. In another subgenus of this aspect of the invention, the dithiophthalide ligand is represented by the following structure:

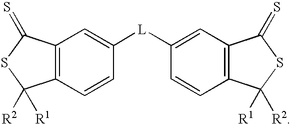

In another subgenus of this aspect of the invention, the dithiophthalide ligand is represented by the following structure:

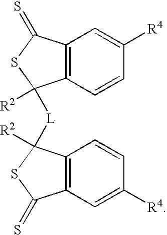

Another aspect of the invention is directed to an assay for a multivalent heavy metal. The assay includes a first step wherein the multivalent heavy metal binds with a dithiophthalide ligand; in the second step, the product of the first step is precipitated for forming a fluorescent dye-doped crystalline analyte; and, in the third step, the fluorescent dye-doped crystalline analyte of the second step is assayed. In a preferred mode of this aspect of the invention, in the third step, the fluorescent dye-doped crystalline analyte of the second step is assayed with a fluorescent microscope. In another preferred mode of this aspect of the invention, in the third step, the assay is performed in a microcapillary tube.

Another aspect of the invention is directed to a process for isolating a multivalent heavy metal ion from a solution. In the first step of the process, the multivalent heavy metal binds with a dithiophthalide ligand; in the second step, the product of the first step is precipitated for forming a fluorescent dye-doped crystalline analyte; and, in the third step, the fluorescent dye-doped crystalline analyte of the second step is isolated.

In summary, an assay for thiophilic heavy metals employs precipitation to decrease interference and increase detection. Analysis in droplets or capillaries provides an effective tool for determining the solubility product of metal complexes using femtomoles of ligand 3a. This assay was conducted with common imaging systems. This finding demonstrates that the combination of ligand synthesis, crystal engineering and fluorescent imaging can provide an information rich platform for toxic metal analyses.

### BRIEF DESCRIPTION OF FIGURES

FIG. 1 illustrates a scheme showing the synthesis of thiophilic ligands 3(a, b) and the structures of established heavy metal indicators 5-7.

FIG. 2 illustrates a three dimensional bar graph that shows the metal ions tested and the solubility products. All the graphs are shaded and the shade of the bars varies along the Y-axis of the graph.

FIG. 3 illustrates six micrographs employable in the microscopic analysis of the metal complexes.

FIG. 4 illustrates six types of generic scaffolds.

FIG. 5 illustrates the synthesis of compounds containing R groups by using a Suzuki reaction.

### DETAILED DESCRIPTION

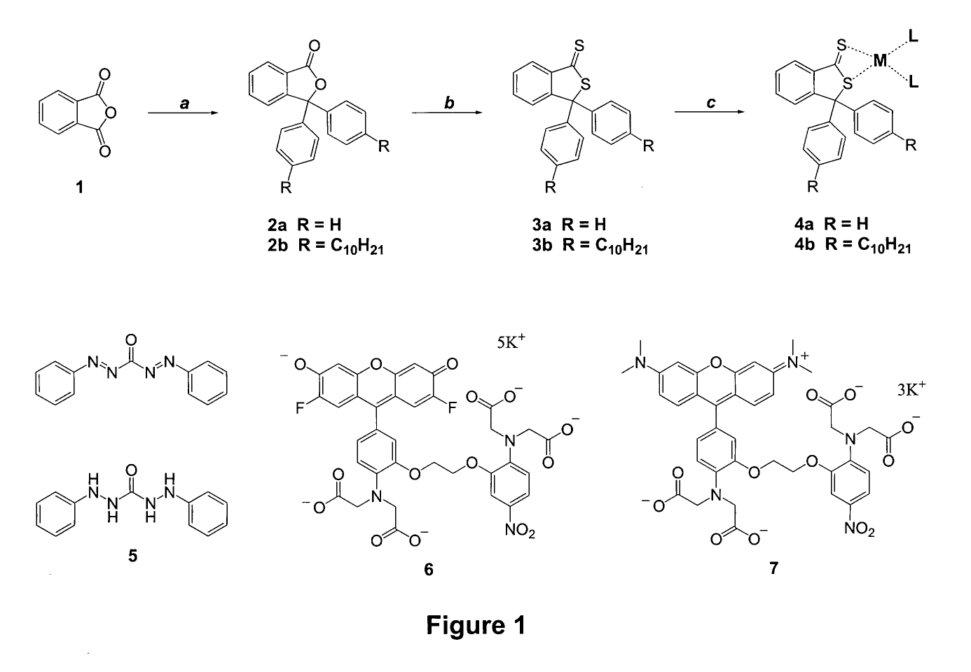

The construction of the assay began with the synthesis of dithiophthalides 3 (Szurdoki, F.; et al. Bioconjugate Chem. 1995, 6, 145-149; Lo, J.-M., et al., Anal. Chem. 1994, 66, 1242-1248; Sachsenberg, S., et al., J. Fresenius Anal. Chem. 1992, 342, 163-166; Bond, A. M., et al., J. Phys Chem. 1991, 95, 7460-7465). As shown in FIG. 1, 3a and 3b were prepared from phthalic anhydride (1) in two operations (Nugara, P. N.; et al. Heterocycles, 1991, 32, 1559-1561; Oparin, D. A.; Kuznetsova, A. S. Vestsi Akademii Navuk BSSR, Seryya Khim. Navuk, 1990, 6, 109-110). Initial cleavage of the anhydride by phenylmagnesium bromide was followed by addition of a second equivalent of phenylmagnesium bromide, and subsequent intramolecular ester formation to give lactone 2a. Compound 2a could then be smoothly converted to dithiophthalide 3a by treatment with P₄S₁₀ in refluxing xylenes. Importantly, the entire route to 3a could be executed at the kg-scale without chromatographic purification.

When presented to a panel of metal ions, 3a complexed and precipitated Hg²⁺, Pb²⁺, Cd²⁺, Au³⁺, Cu²⁺, Pd²⁺, Ni²⁺, Co²⁺, Mo²⁺ and Pt²⁺. Job plots indicated that precipitates 4a (M=Hg²⁺, Pb²⁺, Cd²⁺) existed as a 1:1 complex. A 2:1 ligand to metal complex could also be formed when precipitating from solutions containing >5 mM in 3a. In contrast, the precipitation of 3b by Hg²⁺ and Pb²⁺ required over 5 min for induction, and other metals including Cd²⁺ failed to provide sufficient yields of precipitate. Consequently, we deemed that the solubility of 3b and its metal complexes do not provide a response adequate for analytical use.

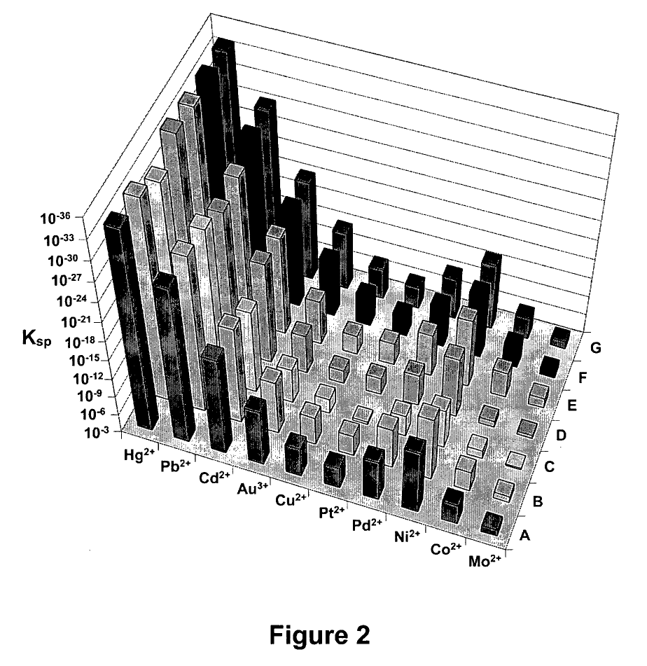

Solubility products were determined using conventional mass analysis to characterize the metal ion selection of 3a (FIG. 2A). Alternatively, the precipitation of 4a could be determined by measuring the spectrophotometric loss of 3a in the supernatant at λₘₐₓ=304, 330 or 356 nm (FIG. 2B). Quantitative analysis with 3a was comparable in accuracy and precision to assays developed with conventional ligands, including diphenylcarbazone 5, Fluo-5N 6 and Rhod-5N 7 (Indicators 5-7 displayed high affinities (K_{d}=10⁻⁹-10⁻¹² M⁻¹) to Hg²⁺, Pd²⁺ and Co²⁺, and permitted detection of micromolar levels of these metal ions in the presence of 10³ equivalents of non-binding metals. See, R. Haugland, Handbook of Fluorescent Probes and Research Products, 9th Edition, Molecular Probes, 2001, Section 20.). The precipitation of 3a in 10% aqueous CH₃CN was visually apparent upon addition of 2 μM Hg(OAc)₂ to 1 μM 3a, 50 μM CdCl₂ to 25 μM 3a, and 10 μM Pb(OAc)₂ to 5 μM 3a. The precipitation of 4a with Hg²⁺, Pb²⁺ or Cd²⁺ was tolerant of alkali metals (Na⁺, K⁺), alkaline earth metals (Ca²⁺, Mg²⁺), and transition metals (Mn²⁺, Mo²⁺, Cr²⁺, and Fe²⁺), as given by the modest change in Kₛₚ when complexes of 4a were formed in the presence of 10⁵ molar excesses of non-binding metal ions (FIG. 2C). This selection permitted the analysis of Hg²⁺, Pb²⁺ and Cd²⁺ in mixtures containing 500 equivalents of Cu²⁺ in contrast to established indicators for Hg²⁺ such as Rhod-5N 6 which typically offer only a 3-5 fold selection for Hg²⁺ over Cu²⁺.

Metal selectivity is not the only criteria required for a practical screen. Many indicators, including 5-7, are sensitive to pH, ion strength, impurities, buffers, and solvents. Deviations in these environmental factors can alter the kinetics of ligand association or the photophysical properties of the appended reporter. These complications are furthered by fact that the concentration profile of many solution-based colorimetric ligands remains non-linear. For instance, the affinity of Fluo-5N 6 to Cd²⁺ is O-fold larger than La³⁺ at 1 μM, while the affinity for the complexes changes to favor La³⁺ by 2-fold over Cd²⁺ at 100 μM (Kuhn, M. A.; et al. Proc. SPEI, Intl. Soc. Opt. Eng. 1995, 2388, 238-242; Indicators 5-7 displayed high affinities (K_{d}=10⁻⁹-10⁻¹² M⁻¹) to Hg²⁺, Pd²⁺ and Co²⁺, and permitted detection of micromolar levels of these metal ions in the presence of 103 equivalents of non-binding metals. See, R. Haugland, Handbook of Fluorescent Probes and Research Products, 9th Edition, Molecular Probes, 2001, Section 20.). The fact that Kₛₚ values (FIG. 2A-C) were reproducible within 1% deviation over a wide range of concentrations (1 mM-0.1 μM), pH (3-10), and temperature (0-50° C.) indicated that the precipitation of 3a offered increased resistance to environmental factors relative to comparable solution-based methodologies. This does not imply, however, that a precipitation-based assay can supplant existing solution technologies for heavy metal detection. Current fluorescent probes that operate in solution are directly amenable to in situ analysis and thus can assay samples that cannot be removed from their natural environment. In contrast, our precipitation-based methodology is very resistant to signal degradation by environmental factors (vide supra), yet requires extraction of the sample from its environment for testing.

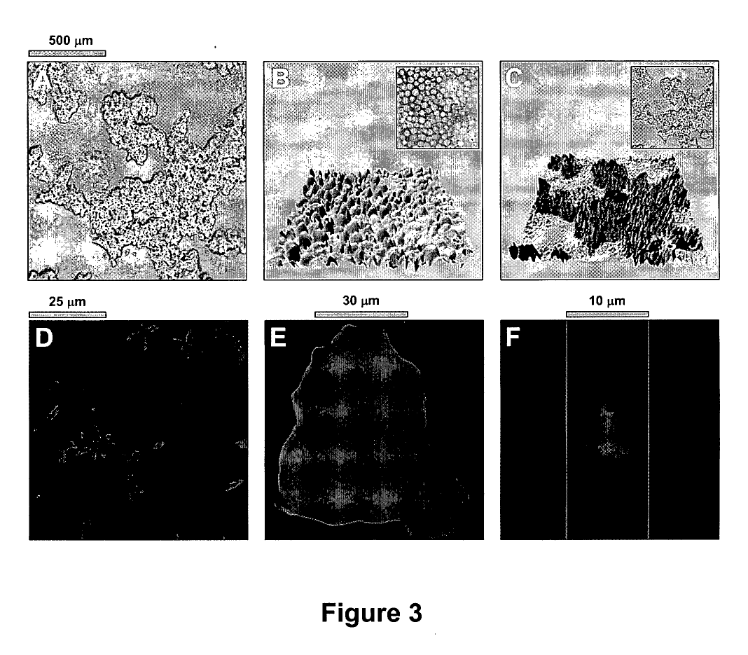

The formation of metal precipitates was rapidly screened using high-throughput video-capture techniques. When conducted on a glass slide or in a fused-silica capillary, crystals 4a (M=Hg²⁺, Pb²⁺ or Cd²⁺) associated on the surface of a glass slide or wall of a capillary within few seconds after formation (FIG. 3). Elemental analysis of the resulting supernatant indicated that the mercuric ion was quantitatively removed from the solution during this process. Indeed, vapor atomic absorption measurements demonstrated that the amount of Hg²⁺ (0.58±0.03 μM) after reacting 6.7±0.01 mM Hg(OAc)₂ with an 6.7±0.01 mM 3a was well below the error threshold of the pipettors used to prepare the reaction (±0.1 l or ±4 μM). To improve visualization, the crystals were doped with a fluorescent dye, as given by the addition of 10⁻³ equivalents of Rhod5N or rhodamine B during precipitation to yield needles (FIG. 3D) or globular crystals (FIG. 3E).

A digital displacement map method was developed to determine the mass of precipitate within each image. A Delaunay triangulation (Wohlberg, B.; de Jager, G. IEEE T image processing 1999, 8, 1716-1729; Lohner, R. Finite Elements Anal. Design 1997, 25, 111-134) was used to transpose each image (FIG. 3) into a 3D vector map (Zhu, W.; et al. Opt. Soc. America A 1999, 14, 799-802). This process provided a net volume of precipitate generated per image using vector analysis. This volume deviated 3.9±1.5% and 6.1±2.2% when imaging standard 100 μm microspheres (FIG. 3B), on an inverted microscope (Nikon Eclipse TE300) or inexpensive CCD microscope (Intel Digital Blue), respectively. Once calibrated with microspheres, the volumes of precipitates were quickly determined from images of precipitates 4a (FIG. 3C inset) and their corresponding 3D maps (FIG. 3C). Using the conditions described in FIG. 303A, the CCD microscope (FIG. 2D) and inverted microscope (FIG. 2E) provided Kₛₚ values that were comparable to that obtained by conventional assays (FIG. 2A-B). Kₛₚ values were calculated based comparing the measured amount of precipitate generated with the known aliquot of metal ion. The amount of precipitate generated was calculated from the volume of precipitate using a density of 7.3, 3.8, and 4.3 g/ml, for the precipitate generated by the addition of 3a to Hg(OAc)₂, Pb(OAc)₂, and CdCl₂, respectively. An average of 20 repetitions was provided.

The method was capable of detecting ppb levels of thiophilic metals when examined in small volume elements. As shown in FIG. 3F, single crystals of 4a were reproducibly generated upon the addition of microcapillaries filled with 3a into aqueous solutions of metal ion. Displacement map analysis indicated that the crystal in FIG. 3F contained 16.5±2.9 femtomoles of 4a. Assuming a 1:1 complex, this finding represented the detection of 0.17±0.3 nM Hg²⁺ (0.3 ppb) and indicated an 82% yield of 4a upon exposure to a 100 μl aliquot of 0.2 nM Hg²⁺. Using the procedure in FIG. 3F, comparable precipitates were obtained when exposed to solutions that contained greater than 0.2 nM (0.4 ppb) Hg²⁺, 1.5 nM (0.31 ppb) Pb²⁺, or 2.5 nM (0.28 ppb) Cd²⁺.

### EXPERIMENTAL

General Methods. Unless otherwise stated, all reactions were performed under an inert atmosphere with dry reagents and solvents and flame-dried glassware. Analytical thin-layer chromatography (TLC) was performed using 0.25 mm pre-coated silica gel Kieselgel 60 F₂₅₄ plates. Visualization of the chromatogram was by UV absorbance, iodine, dinitrophenylhydrazine, ceric ammonium molybdate, ninhydrin or potassium permanganate as appropriate. Preparative and semi-preparative TLC was performed using Merck 1 mm or 0.5 mm coated silica gel Kieselgel 60 F₂₅₄ plates respectively. Methylene chloride and chloroform were distilled from calcium hydride. Tetrahydrofuran (THF) was distilled from sodium/benzophenone. Methanol was distilled from magnesium. ¹H and ¹³C NMR spectra were recorded on a Varian INOVA-399 spectrometer at 400 MHz and 100 MHz respectively and are reported in ppm, unless otherwise noted. All spectra were processed with 0.5 Hz line broadening. Matrix-assisted laser desorption/ionization (MALDI) FTMS experiments are performed on an IonSpec FTMS mass spectrometer. Electrospray ionization (ESI) mass spectrometry experiments were performed on an API 100 Perkin Elmer SCIEX single quadrupole mass spectrometer.

3,3-Diphenylisobenzofuran-1(3H)-one (2a). To a solution of phthalic anhydride (5 g, 33.8 mmol) in benzene (100 mL), phenylmagnesium bromide (84.5 mmol) was added slowly and the solution heated to reflux for 24 h. The reaction was then cooled, and 2M HCl (100 mL) added slowly. The organic phase was separated, washed with water (3×20 mL), dried on MgSO₄, and concentrated. The resulting residue was then dissolved in EtOH (75 mL), hydrazine hydrate (3 mL) was added, and heated to reflux for 24 h. The solution was then cooled to 4° C. and the resulting yellow crystals collected and dried in vacuo to give 3.7 g (38%) of the desired lactone 2a. ¹H NMR (CDCl₃, 300 MHz) δ 7.94 (d, J=7.5 Hz, 1H), 7.69 (t, J=7.2 Hz, 1H), 7.58 (d, J=7.5 Hz, 1H), 7.53 (d, J=7.2 Hz, 1H), 7.33 (m, 10H). ¹³C NMR (CDCl₃, 75 MHz) δ 169.8, 152.0, 141.0, 134.3, 129.5, 128.7, 128.6, 127.3, 126.2, 125.7, 124.4, 92.0. MALDI-FTMS for C₂₀H₁₄O₂ (M+H⁺) calculated 287.1067, found 287.1058.

3,3-Diphenylbenzo[c]thiophene-1(3H)-thione (3a). Lactone 2a (489 mg, 1.71 mmol) was dissolved in xylene (25 mL). To this solution, P₄S₁₀ (380 mg, 0.86 mmol) was added and the reaction heated to reflux for 18 h. The solution was then cooled, filtered, and concentrated to give the desired product 3a in excellent yield (540 mg, 99%). ¹H NMR (CDCl₃, 300 MHz) δ 8.08 (d, J=8 Hz, 1H), 7.59 (dt, J=0.5 Hz, 8 Hz, 1H), 7.48 (dt, J=0.5 Hz, 8 Hz, 1H), 7.30 (m, 11H). ¹³C NMR (CDCl₃, 75 MHz) δ 225.5, 153.0, 142.4, 142.0, 132.9, 128.9, 128.7, 128.5, 128.1, 127.6, 125.3. MALDI-FTMS for C₂₀H₁₄S₂ (M+H⁺) calculated 319.0610, found 319.0609. UVN is (CH₃CN): λₘₐₓ (ε)=509 (1850), 340 (5600), 220 nm (13000).

3,3-Bis(4-decylphenyl)isobenzofuran-1(3H)-one (2b). A solution of 4-decylphenylmagnesium bromide (˜1 M in THF) was prepared from magnesium (90 mg, 3.7 mmol) and 4-decylphenyl bromide (1 g, 3.4 mmol). This Grignard reagent was added to a solution of phthalic anhydride (201 mg, 1.36 mmol) in toluene (10 mL) and the solution heated to reflux for 24 h. The dark red solution was then cooled to room temperature, and quenched with 20% aqueous HCl (10 mL). The organic phase was separated, washed with water (2×10 mL), saturated NaCl (10 mL), and concentrated. The resulting residue was then dissolved in EtOH (10 mL), hydrazine hydrate (1 mL) was added, and heated to reflux for 24 h. The reaction was then cooled to room temperature, concentrated in vacuo, and purified by radial chromatography (5:95-EtOAc:hexane) to give 103 mg (14%) of the desired lactone 2b. ¹H NMR (CDCl₃, 500 MHz) δ 7.93 (dd, J=0.75 Hz, 7.7 Hz, 1H), 7.67 (dt, J=1.1 Hz, 7.7 Hz, 1H), 7.55 (m, 2H), 7.23 (d, J=8.2 Hz, 4H), 7.12 (d, J=8.2 Hz, 4H), 2.58 (t, J=7.5 Hz, 4H), 1.59 (m, 5H), 1.29 (m, 32H), 0.88 (t, J=7 Hz, 6H). ¹³C NMR (CDCl₃, 125 MHz) δ 170.4, 152.9, 143.8, 138.6, 134.5, 129.6, 128.8, 127.5, 126.4, 126.1, 124.6, 92.3, 36.0, 32.4, 31.8, 30.1, 30.0, 29.9, 29.8, 23.2, 14.6. MALDI-FTMS for C₄₀H₅₄O₂ (M+H⁺) calculated 567.4196, found 567.4184.

3,3-Bis(4-decylphenyl)benzo[c]thiophene-1(3H)-thione (3b). Lactone 2b (47 mg, 83.0 μmol) was dissolved in xylene (2 mL). To this solution, P₄S₁₀ (30 mg, 67.6 μmol) was added and the reaction heated to reflux for 18 h. The solution was then cooled, filtered, and concentrated to give the desired product 3b as a red oil. This was further purified by chromatography on silica using hexane as the eluent to give 22 mg (44%). ¹H NMR (CDCl₃, 500 MHz) δ 8.08 (dd, J=0.75 Hz, 8 Hz, 1H), 7.59 (dt, J=0.5 Hz, 8 Hz, 1H), 7.48 (dt, J=0.5 Hz, 8 Hz, 1H), 7.27 (d, J=8.8 Hz, 1H), 7.20 (d, J=8.5 Hz, 4H), 7.11 (d, J=8.5 Hz, 4H), 2.58 (t, J=7.7 Hz, 4H), 1.59 (m, 4H), 1.28 (m, 29H), 0.88 (t, J=7 Hz, 6H). ¹³C NMR (CDCl₃, 125 MHz) δ 226.0, 153.4, 142.8, 142.3, 139.0, 132.7, 128.5, 128.2, 127.4, 125.0, 35.5, 31.9, 31.3, 29.6, 29.5, 29.4, 29.3, 22.7, 14.1. MALDI-FTMS for C₄₀H₅₄S₂ (M+H⁺) calculated 599.3739, found 599.3742.

General procedure for thiophilic metal precipitation. A 10 μL aliquot of a 200 mM stock of metal ion in water was incubated at room temperature with 200 μL of a 10 mM stock of ligand 3a in CH₃CN. Red precipitates from 3a appeared upon increasing the amount of water in the final reaction mixture to over 20% (v/v). The formation of these red precipitates provided an ideal tool for verification of sample quality. Red precipitates from 3a were readily removed by triturating the precipitate with acetonitrile.

The lack in solubility of the complexes of 4a (M=Hg²⁺, Pb²⁺ and Cd²⁺) not only offered metal selection, but also allowed one to increase the selectivity of the method as trituration could be used to remove more soluble complexes. For instance, Mo²⁺ complexes of 3a were readily extracted from Hg²⁺ complexes 4a by trituration with hot [?] in 10% acetic acid. Spectroscopic analyses indicated that the rate of precipitation did not correlate with the Kₛₚ, and was as given by Hg²⁺>Pb²⁺>Cd²⁺>(Au³⁺˜Cu²⁺)>(Pd²⁺˜Ni²⁺)>(Co²⁺˜Mo²⁺˜Pt²⁺).

Determination of the extent of metal precipitation. An 167 μL aliquot of a 20 mM stock of Hg(OAc)₂ in water was incubated at room temperature with 333 μL of a 10 mM stock of ligand 3 in CH₃CN. Precipitation occurred immediately. After 5 minutes the sample was centrifuged for 5 min at 14,000×g. The supernatant was removed and an aliquot submitted to vapor atomic absorption analysis (Galbraith Laboratories, Knoxyille, Tenn.).

Determination of solubility products (Kₛₚ). Weight analysis (FIG. 2A): Kₛₚ values were determined either by weighing the amount of precipitate 4a. Scale-up was required to provide sufficient material for analysis on conventional microbalances. The following procedure was used for this analysis: a 100 μL aliquot of a 200 mM stock of metal ion in water was incubated at room temperature with 2 mL of a 10 mM stock of ligand 3a in acetonitrile. After 10 minutes the precipitate was isolated by centrifugation for 5 min at 2,000×g, washed with acetonitrile (5 ml), methanol (5 ml) and dried in vacuo. Spectrophotometric analysis (FIG. 2B): A 10 μL aliquot of a 200 mM stock of metal ion in water was added to 200 μL of a 10 mM stock of ligand 3a in acetonitrile in a spin filter (Millipore). After 10 minutes at rt, the precipitate was removed by centrifugation at 2,000×g. Spectroscopic analysis of the supernatant was performed on a conventional microarray reader (PerSeptive Biosystems CytoFluor or Perkin Elmer HST 7000 plate reader).

Heavy metal competitions. A 167 μL aliquot of a 20 mM stock of metal ion in water was incubated at room temperature with 333 μL of a 10 mM stock of ligand 3a in acetonitrile. After 10 minutes the sample was centrifuged for 5 min at 2,000×g or filtered through a 0.8 μm filter plate. The supernatant was analyzed on a PerSeptive Biosystems CytoFluor or Perkin Elmer HST 7000 plate reader using excitation at 510 nm. The metals presented were prepared using LiCl (EM Chemicals OmniPure), NaCl (Baker), KCl (EM Chemicals OmniPure), CsCl (Aldrich), MgCl₂.6H₂O (EM Chemicals Omni Pure), CaCl₂.2H₂O (EM Chemicals OmniPure), Ba(OAc)₂ (Alfa AESAR), VCl₃ (Alfa AESAR), CrCl₃.6H₂O (EMD Chemicals), MoCl₃ (Alfa AESAR), Mn(OAc)₂ (Alfa AESAR), FeCl₃.6H₂O (EMD Chemicals), CoCl₂.6H₂O (Aldrich), RhCl₃ (Alfa AESAR), NiCl₂.6H₂O (Aldrich), PdCl₂ (Alfa AESAR), PtCl₂ (Alfa AESAR), CuCl₂ (EMD Chemicals), AgCl (Aldrich), AuCl₃ (ICN), ZnCl₂ (EMD Chemicals), CdCl₂ (EMD Chemicals), HgCl₂ (Aldrich), Al(OAc)₃ (Alfa AESAR), Sn(OAc)₂ (Alfa AESAR), Pb(OAc)₂.3H₂O (EMD Chemicals), AsCl₃ (Aldrich), SbCl₃ (Alfa AESAR), BiCl₂ (Alfa AESAR), La(OAc)₃ (Aldrich), CeCl₃ (Aldrich), Sm(OAc)₃ (Aldrich), Eu(OAc)₃ (Aldrich), and Yb(OAc)₃ (Aldrich).

Fluorescent doping of precipitation reactions. Fluorescent complexes 4a were prepared by the addition of 200 mM metal in water to 1 mM 3a in the presence of 5 μM Rhod5N (Molecular Probes R-14207) or 5 μM rhodamine B. Advantageously, the addition of Rhod5N or rhodamine B led to the formation of needles or globular crystals. Doping the reactions in this manner reduced the amount of dye required while providing sufficient fluorescence for analysis on a fluorescence microscope (Nikon Eclipse TE300). After aggregating on the glass surface, the precipitate was then washed with H₂O (3×300 μL). A Nikon Eclipse TE300 was used for this study. White light images were collected using Hoffman Modulation Contrast at 100× or 1000×. Fluorescent images were collected using Y-2E/C (560BP40 excitation and 595 LP 630/60 BP emission) filter.

Displacement map analysis. Positioning of the 1000 regions of each well was regulated by the assistance of an XY stage (XY stage 85-16, Linos). Precise movements (±5 μm) about the surface of the well were regulated by mounting the sample on the XY stage and attaching this stage to the microscope using a small (10 cm²) microbench. A digital micrometer could be added for automation. The predicted volume was calculated by multiplying the number of microspheres by bead volume. Microspheres such as 30-100 μm polymethyl methacrylate (Sigma) or polystyrene microspheres (Sigma) were routinely for this analysis.

While analytical determinations of precipitation can be preformed in laboratory settings using microbalances or spectroscopic analysis, such machines are far too expensive for remote applications. To solve this problem, we developed a digital displacement routine to determine the volume, and hence the weight, of precipitate. This technique was based on conventional 3D volume analysis tools that use Delaunay triangulation projections to generate at 3D mesh from a 2D image. The volume of the resulting 3D meshes was determined using conventional space filling algorithms. As shown in FIGS. 3A-B, 3D-meshes provide an accurate representation of their 2D microscopic image.

The accuracy of this method was established by comparing a simple CCD camera (Digital Blue) to an inverted microscope (Nikon Eclipse TE300). Assays were conducted in wells (surface area of 0.9 cm²) of a chamber slide system (Lab-Tek*). To reduce optical errors, the bottom of each reaction well was divided into 1000 individual 300 μm×300 μm regions and each region was filmed at 20 frames for 5 seconds. Each film was compiled into a single image using a scatter correction algorithm and processed using displacement map analysis. The volume of precipitate found in each region was tabulated and the amount of precipitate found per assay was determined by summation. Calibrations using 100 μm microspheres indicated that the Digital Blue camera provided only modest accuracy, delivering a volume of precipitate that deviated within 6% of the predicted volume, as compared to 4% for the inverted microscope. This method was then used to determine the Kₛₚ of metal complexes 4a (Table 1, column D-E). The error in the Kₛₚ the CCD camera was only modestly greater that obtained with the inverted microscope. Although this error was considerable, the method was far more sensitive than visual analysis, and thereby provided an effective system for analyses in non-laboratory settings.

Capillary analysis of heavy metal precipitation. A 50 μL aliquot of a stock solution of Hg(OAc)₂ (10 mM) and Rhod-5N (50 μM; Molecular Probes R-14207) in H₂O was added to 100 μL of a 10 mM ligand 3a stock in CH₃CN. Immediately after mixing, a sample of this solution was loaded into a 1 mm long segment of a 10 μm ID capillary (Polymicro Technologies LTD). The loading was conducted under a microscope to ensure that crystal formation occurred after reaction within the capillary. After incubation at room temperature for 15 min, the capillary was imaged using both an inverted and conventional fluorescent microscopes.

The material requirements of this assay were reduced using micron-sized capillaries. Fluorescent crystals of 4a (M=Hg) were generated in capillaries by placing a 1±0.05 mm long section of 10 μm ID Fused silica capillary (Polymicro Technologies Inc.) containing 200 μM 3a and 2 μM rhodamine B in acetonitrile into a 100 μl aliquot of aqueous metal ion. Single or multiple crystals of 4a were generated by placing capillaries loaded with 3a and dye into aqueous solutions containing 0.2 nM Hg²⁺. Using displacement map analysis, the capillary used for the experiment contained 16.5±2.9 femtomoles of 4a. Assuming a 1:1 metal to ligand complex in 4a, this represented the detection of 0.17±0.3 nM Hg²⁺ (0.3 ppb). This finding indicated an 82% yield of 4a upon exposure to a 100 μl aliquot of 0.2 nM Hg²⁺. Using the same capillaries, precipitates were obtained when exposed to solutions that contained greater than 0.2 nM (0.4 ppb) Hg²⁺, 1.5 nM (0.31 ppb) Pb²⁺, or 2.5 nM (0.28 ppb) Cd²⁺.

### DETAILED DESCRIPTION OF FIGURES

FIG. 1 is a scheme showing the synthesis of thiophilic ligands 3 and the structures of established heavy metal indicators 5-8. Steps required in the synthesis: a) i. PhMgBr, C₆H₆, 80° C., 24 h; ii. 2M HCl; iii. N₂H₄.H₂O, EtOH, 78° C., 24 h. b) P₄S₁₀, xylene, 140° C., 18 h. c) Mn⁺ (OAc)ₙ or Mn⁺ Cln, H₂O, CH₃CN, <1 min.

FIG. 2 is a three dimensional bar graph that shows the metal ions tested and the solubility products. All the graphs are in color and the color of the bars varies along the Y-axis of the graph. Solubility products (Kₛₚ) of metal complexes of 3a as determined by: A) mass of precipitate 4a, B) spectroscopic loss of 3a, C) mass of 4a obtained in the presence of 10⁵ molar equivalents of Li⁺, Na⁺, K⁺, Cs⁺, Mg²⁺, Ca²⁺, Ba²⁺, V²⁺, Cr²⁺, Mn²⁺, Fe²⁺, Co²⁺, Rh³⁺, Zn²⁺, Al³⁺, Sn²⁺, As³⁺, Sb³⁺, Bi³⁺, La³⁺, Ce³⁺, Sm³⁺, Eu³⁺, and Yb³⁺. The solubility product could also be determined using a digital displacement map analysis as given by imaging: D) with a CCD microscope (Intel Digital Blue), E) on an inverted microscope (Nikon Eclipse TE300), F) fluorescence crystals of 4a (FIG. 3E); G) fluorescent crystals 4a grown in capillaries (FIG. 3G).

FIG. 3 shows six images which are used in the microscopic analysis of the metal complexes. Microscopic analysis. A) The precipitate generated by mixing 2 μM Hg(OAc)₂ with 1 μM 3a in 10% aqueous CH₃CN, B) An image (inset) and the Delaunay triangulation map of 100 μm microspheres, C) An image (inset) and Delaunay map of precipitates 4a (M=Hg), D) Fluorescent microcrystals 4a generated by the mixing of 1 mM Hg(OAc)₂, 1 mM 3a and 0.5 μM Rhod-5N in 10% aqueous CH₃CN, E) Globular crystals formed from 20 mM Hg²⁺, 20 mM 3a and 0.5 μM Rhod-5N, F) A single fluorescent crystal of 4a (M=Hg) was generated in capillaries by placing a 1±0.05 mm long section of 10 μm ID Fused silica capillary containing 200 μM 3a and 2 μM rhodamine B in CH₃CN into a 100 μl aliquot of 0.2 nM Hg(OAc)₂. Images were collected on a Nikon Eclipse TE300 using Y-2E/C (560BP40 excitation and 595 LP 630/60 BP emission) filter. Comparable images were also generated when precipitating with equimolar amounts of Pb(OAc)₂, Cd(OAc)₂, HgCl₂, PbCl₂, and CdCl₂.

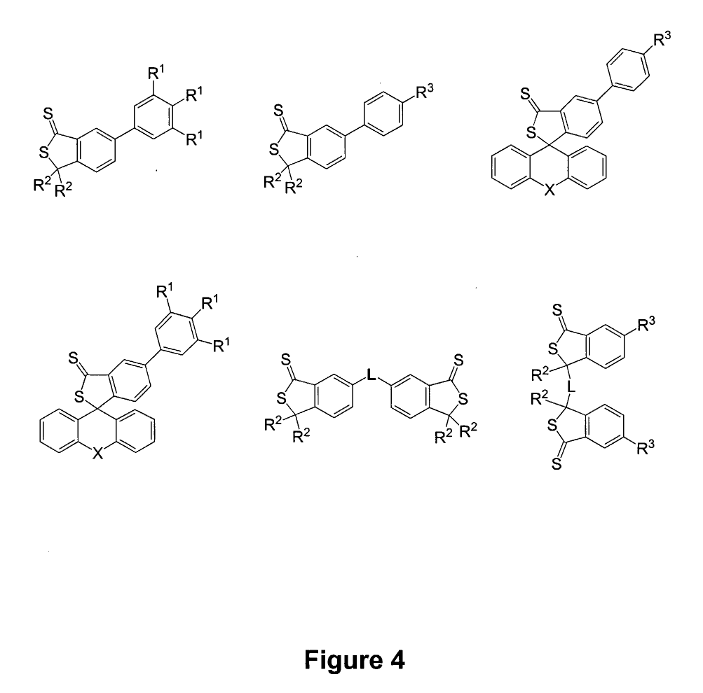

FIG. 4 shows the structures of 6 different generic scaffolds. R¹=—H, —NO₂, —NH₂, —OH, —CO₂H, —CO₂Me, —CO₂t-Bu, —CH₂OH, —CHO, —C(O)CH₃, —Cl, —Br, —I, —CF₃, —CN, —CH═CH₂, aryl, or alkyl substituents; R²=aryl or alkyl substituents; R³=—H, —Cl, —Br, —I; L=alkyl, aryl, polyethylene glycol, peptide, oligonucleotide linker; X=—O—, —NH—, —C(O)—, —C(S)—.

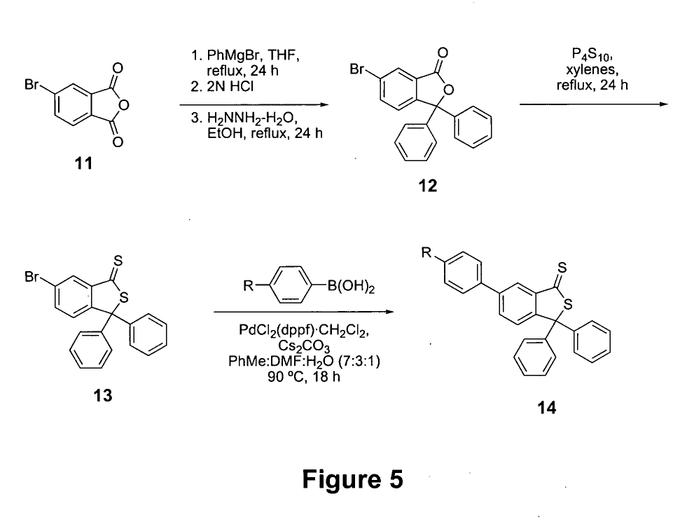

FIG. 5 shows a scheme for the synthesis of the thiophilic ligands where R is not hydrogen. The starting material is 4-bromophthalic anhydride or 5-bromoisobenzofuran-1,3-dione which is reacted with an excess of phenylmagnesium bromide. The lactone is purified from unreacted anhydride and ketones by reaction with hydrazine hydrate. Crystallization of the mother liquor gives the desired product. Reaction with phosphorus pentasulfide in refluxing xylenes gives the red-solid, 13. A Suzuki coupling with the desired substituted phenylboronic acid using a catalytic amount of PdCl₂(dppf).CH₂Cl₂ and two equivalents of Cs₂CO₃ in toluene:DMF:H₂O.

1. A thiophilic metal-ligand complex represented by formula I: 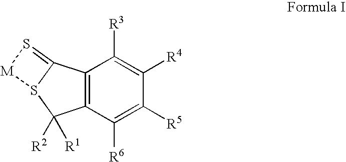
wherein:
M is a multivalent heavy metal ion;
R¹ and R² are each radicals independently selected from the group consisting of C1-C10 alkyl, C6-C10 aryl, C5-C10 heteroaryl, and a radical represented by the following structure: 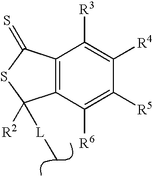
or alternatively, R¹ and R² together form a diradical represented by formula II: 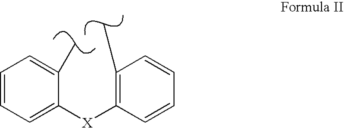
R³, R⁵, and R⁶ are each radicals independently selected from the group consisting of hydrogen, —NO₂, —NH₂, —OH, —CO₂H, —CO₂Me, —CO₂t-Bu, —CH₂OH, —CHO, —C(O)CH₃, —Cl, —Br, —I, —CF₃, —CN, —CH═CH₂, C1-C10 alkyl, C6-C10 aryl, and C5-C10 heteroaryl; and
R⁴ is a radical selected from the group consisting of hydrogen, —NO₂, —NH₂, —OH, —CO₂H, —CO₂Me, —CO₂t-Bu, —CH₂OH, —CHO, —C(O)CH₃, —Cl, —Br, —I, —CF₃, —CN, —CH═CH₂, C1-C10 alkyl, C6-C10 aryl, C5-C10 heteroaryl, and either radical represented by the following structures: 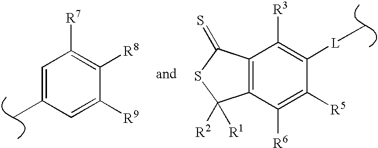
wherein R⁷, R⁸, and R⁹ are each radicals independently selected from the group consisting of hydrogen, —NO₂, —NH₂, —OH, —CO₂H, —CO₂Me, —CO₂t-Bu, —CH₂OH, —CHO, —C(O)CH₃, —Cl, —Br, —I, —CF₃, —CN, —CH═CH₂, C1-C10 alkyl, C1-C10 aryl, and C5-C10 heteroaryl;
X is a diradical selected from the group consisting of —O—, —NH—, —C(O)—, and —C(S)—; and
L is a diradical selected from the group of diradicals consisting of C1-C10 alkyl, C6-C10 aryl, C5-C10 heteroaryl, polyethylene glycol having 1-10 subunits, peptide having 1-10 amino acid residues, and oligonucleotide having 1-10 nucleotide residues.

2. A thiophilic metal-ligand complex according to claim 1 wherein M is a multivalent metal ion selected from the group consisting of Hg⁺⁺, Pb⁺⁺, Cd⁺⁺, Au⁺⁺⁺, Cu⁺⁺, Pt⁺⁺, Pd⁺⁺, Ni⁺⁺, Co⁺⁺, and Mo⁺⁺.

3. A thiophilic metal-ligand complex according to claim 1 wherein R⁴ is a radical selected from the group consisting of —H, —Cl, —Br, and —I.

4. A thiophilic metal-ligand complex according to claim 1 wherein R⁸ is a radical selected from the group consisting of —H, —Cl, —Br, and —I.

5. A thiophilic metal-ligand complex according to claim 1 represented by the following structure:

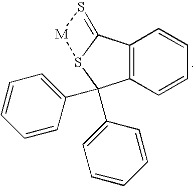

6. A thiophilic metal-ligand complex according to claim 1 represented by the following structure:

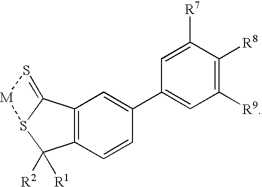

7. A thiophilic metal-ligand complex according to claim 6 wherein R⁷ and R⁹ are hydrogen and R⁸ is a radical selected from the group consisting of —H, —Cl, —Br, and —I.

8. A thiophilic metal-ligand complex according to claim 1 represented by the following structure:

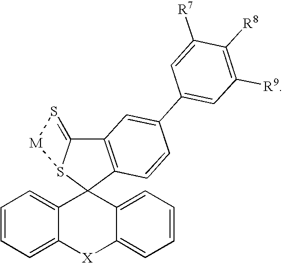

9. A thiophilic metal-ligand complex according to claim 8 wherein R⁷ and R⁹ are hydrogen and R⁸ is a radical selected from the group consisting of —H, —Cl, —Br, and —I.

10. A thiophilic metal-ligand complex according to claim 1 represented by the following structure:

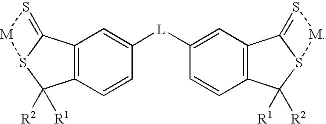

11. A thiophilic metal-ligand complex according to claim 1 represented by the following structure:

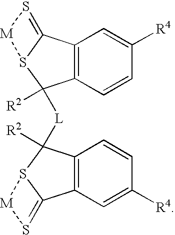

12. A precipitate of a thiophilic metal-ligand complex according to claim 1.

13. A dithiophthalide ligand represented by formula III: 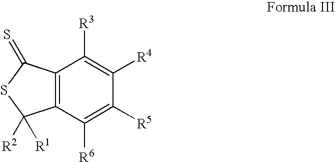
wherein:
R¹ and R² are each radicals independently selected from the group consisting of C1-C10 alkyl, C6-C10 aryl, C5-C10 heteroaryl, and a radical represented by the following structure: 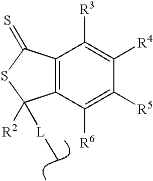
or alternatively, R¹ and R² together form a diradical represented by formula IV: 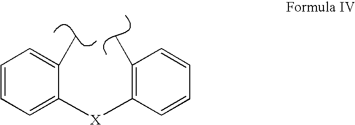
R³, R⁵, and R⁶ are each radicals independently selected from the group consisting of hydrogen, —NO₂, —NH₂, —OH, —CO₂H, —CO₂Me, —CO₂t-Bu, —CH₂OH, —CHO, —C(O)CH₃, —Cl, —Br, —I, —CF₃, —CN, —CH═CH₂, C1-C10 alkyl, C6-C10 aryl, and C5-C10 heteroaryl; and
R⁴ is a radical selected from the group consisting of hydrogen, —NO₂, —NH₂, —OH, —CO₂H, —CO₂Me, —CO₂t-Bu, —CH₂OH, —CHO, —C(O)CH₃, —Cl, —Br, —I, —CF₃, —CN, —CH═CH₂, C1-C10 alkyl, C6-C10 aryl, C5-C10 heteroaryl, and either radical represented by the following structures: 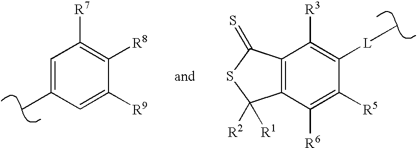
wherein:
R⁷, R⁸, and R⁹ are each radicals independently selected from the group consisting of hydrogen, —NO₂, —NH₂, —OH, —CO₂H, —CO₂Me, —CO₂t-Bu, —CH₂OH, —CHO, —C(O)CH₃, —Cl, —Br, —I, —CF₃, —CN, —CH═CH₂, C1-C10 alkyl, C6-C10 aryl, and C5-C10 heteroaryl;
X is a diradical selected from the group consisting of —O—, —NH—, —C(O)—, and —C(S)—; and
L is a diradical selected from the group of diradicals consisting of C1-C10 alkyl, C6-C10 aryl, C5-C10 heteroaryl, polyethylene glycol having 1-10 subunits, peptide having 1-10 amino acid residues, and oligonucleotide having 1-10 nucleotide residues;
with the following provisos
R⁴ is hydrogen only if R¹ or R² is a radical represented by the following structure: 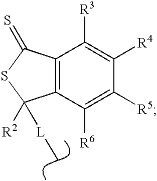
or alternatively, if R¹ and R² together for a diradical represented by formula II: 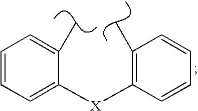
and
R⁴ and R⁵ can not together form a diradical.

14. A dithiophthalide ligand according to claim 13 wherein R⁴ is a radical selected from the group consisting of —H, —Cl, —Br, and —I.

15. A dithiophthalide ligand according to claim 13 wherein R⁸ is a radical selected from the group consisting of —H, —Cl, —Br, and —I.

16. A dithiophthalide ligand according to claim 13 represented by the following structure:

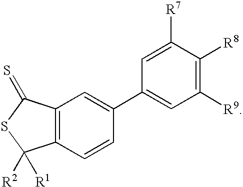

17. A dithiophthalide ligand according to claim 16 wherein R⁷ and R⁹ are hydrogen and R⁸ is a radical selected from the group consisting of —H, —Cl, —Br, and —I.

18. A dithiophthalide ligand according to claim 13 represented by the following structure:

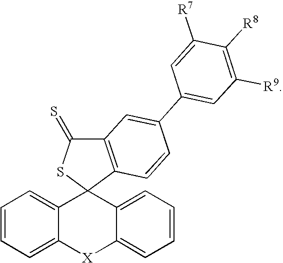

19. A dithiophthalide ligand according to claim 18 wherein R⁷ and R⁹ are hydrogen and R⁸ is a radical selected from the group consisting of —H, —Cl, —Br, and —I.

20. A dithiophthalide ligand according to claim 13 represented by the following structure:

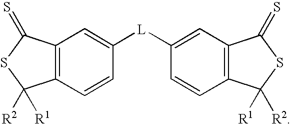

21. A dithiophthalide ligand according to claim 13 represented by the following structure:

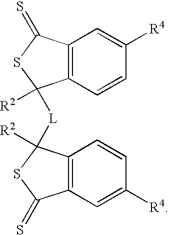

22. An assay for a multivalent heavy metal comprising the following steps:
Step A: binding the multivalent heavy metal with a dithiophthalide ligand;
Step B: precipitating the product of said Step A for forming a fluorescent dye-doped crystalline analyte; and then
Step C: assaying the fluorescent dye-doped crystalline analyte of said Step B.

23. An assay according to claim 22 wherein, in said Step C, a fluorescent dye-doped crystalline analyte of said Step B is assayed with a fluorescent microscope.

24. An assay according to claim 23 wherein, in said Step C, the assay is performed in a microcapillary tube.

25. A process for isolating a multivalent heavy metal ion from a solution, the process comprising the following steps:
Step A: binding the multivalent heavy metal with a dithiophthalide ligand;
Step B: precipitating the product of said Step A for forming a fluorescent dye-doped crystalline analyte; and then
Step C: isolating the fluorescent dye-doped crystalline analyte of said Step B from the solution.
